(19) United States
(12) Patent Application Publication
(10) Pub. No.: US 2018/0085186 A1
(43) Pub. Date: Mar. 29, 2018

(54) Structurally Encoded Rods and Screws

(71) Applicants: Brian Kieser, San Antonio, TX (US); Thomas Zink, San Antonio, TX (US); Nicholas M. Cordaro, Vista, CA (US)

(72) Inventors: Brian Kieser, San Antonio, TX (US); Thomas Zink, San Antonio, TX (US); Nicholas M. Cordaro, Vista, CA (US)

(21) Appl. No.: 15/805,317

(22) Filed: Nov. 7, 2017

Related U.S. Application Data
(63) Continuation-in-part of application No. 14/302,133, filed on Jun. 11, 2014.
Application No. 14/302,133 is a continuation-in-part of application No. 14/456,665, filed on Aug. 11, 2014.
(60) Provisional application No. 62/419,364, filed on Nov. 8, 2016.
(60) Provisional application No. 62/419,341, filed on Nov. 8, 2016.
(60) Provisional application No. 62/419,353, filed on Nov. 8, 2016.
(60) Provisional application No. 62/419,373, filed on Nov. 8, 2016.
(60) Provisional application No. 61/938,475, filed on Feb. 11, 2014.
(60) Provisional application No. 61/938,475, filed on Feb. 11, 2014.

(51) Int. Cl. A61B 90/94; A61B 17/70; A61B 90/98

(52) U.S. Cl. CPC A61B 90/94; A61B 17/7002; A61B 2090/374; A61B 17/7032; A61B 90/98

(57) ABSTRACT

An implant device, such as a screw or a rod, which is identifiable after implantation and comprises a main portion of the implant device and a coded portion of the implant device. The coded portion comprises a radiopaque element and indicia on the radiopaque element or indicia as a plurality of radiopaque elements disposed within the coded portion around an entire circumference of the implant device. The indicia are discernible by x-ray, fluoroscopy, computed tomography, electromagnetic radiation, ultrasound, or magnetic resonance imaging.

### RELATED APPLICATION DATA

This application incorporates by reference all of the following in their entirety: (i) U.S. Provisional Application No. 61/938,475, filed Feb. 11, 2014; (ii) U.S. patent application Ser. Nos. 14/302,133, 14/302,171 (now U.S. Pat. No. 9,101,321) and 14/302,197, all filed Jun. 11, 2014; (iii) U.S. patent application Ser. No. 14/456,665, filed Aug. 11, 2014; (iv) U.S. Provisional Application No. 62/035,875, filed Aug. 11, 2014; (v) U.S. patent application Ser. No. 14/823,234, filed Aug. 11, 2015 (now U.S. Pat. No. 9,424,503); (vi) U.S. patent application Ser. No. 14/822,613, filed Aug. 10, 2015 (now U.S. Pat. No. 9,414,891); (vii) U.S. Provisional Application No. 62/204,233, filed Aug. 12, 2015; U.S. patent application Ser. No. 15/235,914, filed Aug. 12, 2016; (viii) U.S. Provisional Application No. 62/419,373, filed Nov. 8, 2016 and entitled Method of Producing Elongate Implant Containing A Structurally Encoded Pin Through Electrical Discharge Machining; (ix) U.S. Provisional Application No. 62/419,341, filed Nov. 8, 2016 entitled Elongate Implant Containing A Structurally Encoded Pin, Carrier And Reading System Therefor; (x) U.S. Provisional Application No. 62/419,353, filed Nov. 8, 2016; and (xi) U.S. Provisional Application No. 62/419,364, filed Nov. 8, 2016 entitled Optical Image Vertebral Implant Cage and Reading System Therefor. All of the forgoing are hereby incorporated by reference in their entirety.

### CROSS-REFERENCE

This application claims priority from Provisional Patent Application Ser. Nos. 62/419,364, 62/419,341, 62/419,353 and 62/419,373, all of which were filed on Nov. 8, 2016. This application is also a continuation in part of U.S. patent application Ser. Nos. 14/302,133 (filed on Jun. 11, 2014) and 14/456,665 (filed on Aug. 11, 2014), both of which claim priority to Provisional Patent Application Ser. No. 61/938,475 filed on Feb. 11, 2014.

### FIELD OF THE INVENTION

The present invention relates generally to identifiable implantable rods and screws and, in particular, structurally encoded titanium screw assemblies.

### BACKGROUND

Medical implant devices used in surgical procedures, such as surgical rods and screws, can be associated with particular information to guide medical professionals before and after the surgical procedure. Each implant device carries a wealth of information that is valuable to the patient, the implant manufacturer, medical researchers, healthcare professionals, and medical facilities. However, the information, which may include the implant manufacturer and manufacturer's lot number, the data and location of surgical implantation, the responsible surgeon, any medical notes, photographs, or diagrams relating to the implant, surgery, or condition, may not be adequate, properly recorded, or readily accessible for beneficial use by a healthcare professional, implant manufacturer or medical researcher after implantation. Problems relating to poor implant records can lead to unnecessary delay or even medical error by healthcare professionals. Moreover, there are many different implant identification methods currently in place instead of a common system to allow manufacturers, distributors, and healthcare facilities and professionals to effectively track, identify, and manage implant devices and medical device recalls. The U.S. Food and Drug Administration recently announced a program focusing on requirements for unique device identifiers for every medical implant device to address the need for a more robust implant device identification system, the details of which are hereby incorporated by reference here: www.fda.gov/udi, as of the filing date of this application.

Consequently, there is a long felt need in the art for a robust implant device identification system that enables a provider to quickly and un-invasively retrieve information from an implanted device, such as a surgical screw. There is also a long felt need for a structurally encoded implant device, such as a surgical rod and/or screw that protects patient privacy. Finally, there is a long felt need for a structurally encoded device that accomplishes all of the forgoing objectives, and that is relatively inexpensive to manufacture and safe and easy to use.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a rod or a screw device, such as a titanium screw, that is identifiable after implantation and comprises a main portion and a readable portion. The readable portion may comprise a readable element, such as a radiopaque element, and indicia disposed on at least one surface thereof or disposed within the screw. The indicia may include a plurality of modifications to at least one surface of the readable element or a plurality or readable elements disposed within the readable portion such that the indicia are discernible by any medical imaging modality, such as at least one of x-ray, fluoroscopy, computed tomography, electromagnetic radiation, ultrasound, and magnetic resonance imaging.

In accordance with further aspects of the invention, the readable portion may be integral with the main portion. The readable portion may also be disposed within the main portion. The plurality of modifications may include an array of holes in the at least one surface of the readable element. The plurality of modifications may include an array of notches or variations of density in the at least one surface of the readable element. The array of notches in the at least one surface of the readable element may form at least one bar code. The at least one bar code may comprise a Hamming code. The plurality of modifications may be less than or equal to two centimeters in length, and can vary in width, depth and shape.

In accordance with further aspects of the invention, a system for identifying a rod or screw device is provided comprising a device comprising a main portion and a readable portion. The readable portion may comprise a readable element and indicia disposed on at least one surface thereof. The indicia may include a plurality of modifications to at least one surface of the readable element or a plurality of readable elements disposed within the readable portion. The indicia may further include a database containing a plurality of records associated with a plurality of implant devices and a user interface comprising means for displaying information associated with the indicia based on the plurality of records. The indicia may be discernible by at least one of x-ray, fluoroscopy, ultra-sound computed tomography, electromagnetic radiation, ultrasound, and magnetic resonance imaging.

In accordance with further aspects of the invention, a method of identifying a micromanufactured rod or screw device is provided comprising discerning indicia by at least one of x-ray, fluoroscopy, computed tomography, electromagnetic radiation, ultrasound, and magnetic resonance imaging. The indicia may include a plurality of modifications to at least one surface of a readable element or a plurality of readable elements disposed within a readable portion. The method of identifying a micromanufactured rod or screw device may further comprise accessing a plurality of records associated with at least one of a plurality of rod or screw devices and providing information associated with the micromanufactured rod or screw based on the indicia and the plurality of records. As used herein, the term “micromanufactured” encompasses all microfabrication techniques such as additive manufacturing and micromachining, and use of this term is not intended to limit the size or scale constraints or the type of the manufacturing process in any way. The term is used to elucidate the desire for the detectable portion of the rod or screw device of the present invention to be an incorporated portion of said rod or screw.

In accordance with further aspects of the invention, the method of identifying a micromanufactured rod or screw device may further comprise displaying information associated with the micromanufactured rod or screw based on the indicia and the plurality of records through a user interface. The readable portion may be disposed upon a main portion of the micromanufactured device. The plurality of modifications may include an array of holes in the at least one surface of the readable element. The plurality of modifications may include an array of notches in the at least one surface of the readable element. The array of notches in the at least one surface of the readable element may form at least one bar code. The at least one bar code may comprise a Hamming code or other similar methods for error detection and correction that are known in the coding theory art. Additionally data compression may be used in the coded indicia of the preferred embodiment.

In accordance with further aspects of the invention, a rod or screw device identifiable after implantation is provided that comprises a main portion and a readable portion. The readable portion may comprise a plurality of laminae or laminar planes (a finite planar volume). Each of the laminae, hereafter referred to as “laminar planes,” may comprise separately readable indicia such that the indicia may be discernible in three dimensions by at least one of x-ray, fluoroscopy, computed tomography, electromagnetic radiation, ultrasound, and magnetic resonance imaging.

The readable portion may be integral with the main portion. Also, the readable portion may be disposed within the main portion. Further, the indicia may include an array of voids on or in a corresponding laminar plane of the readable portion. The indicia may include an array of embedded markers on or in a corresponding laminar plane of the readable portion. The embedded markers may comprise a modulation of material compositions such that a first material composition of at least one first embedded marker is different than a second material composition of at least one second embedded marker. Further, the indicia may include a first array of embedded markers on or in a first laminar plane of the readable portion and a second array of embedded markers on or in a second laminar plane of the readable portion. The first array may comprise a first embedded marker having a first material composition different than a second material composition of a second embedded marker disposed in the second array. The indicia may comprise information in the form of a code. The code may comprise a Hamming code or other similar methods for error detection and correction that are known in the coding theory art. Additionally, data compression may be used in the coded indicia of the preferred embodiment.

In accordance with further aspects of the present invention, a method of manufacturing an identifiable rod or screw device is provided comprising providing a main portion of the device, providing a readable portion of the device, printing a first material onto a first readable portion surface to create a first printed layer, and printing a second material onto the first printed layer to create a second printed layer. The printing of the first material onto the first readable portion surface or the printing of the second material onto the first printed layer may comprise printing encoded indicia. The encoded indicia may comprise voids in the first material or measurable variations in density. The method of manufacturing an identifiable rod or screw device may further comprise printing a second material onto at least one of the first readable portion surface and the first printed layer, such that the encoded indicia comprises the second material.

In accordance with further aspects of the present invention, a rod or screw device identifiable after implantation is provided that comprises a main portion and a readable portion. In this embodiment, the readable portion comprises an internal structure inside the readable portion. The internal structure comprises a plurality of linking structures. Each of the linking structures has a predetermined size or orientation. The linking structures are interconnected to substantially form the internal structure. The linking structures form predetermined indicia such that the indicia are discernible by any medical imaging modality, such as x-ray, fluoroscopy, computed tomography, electromagnetic radiation, ultrasound, and/or magnetic resonance imaging. The readable portion may be integral with the main portion or disposed upon the main portion. The indicia in the readable portion of the rod or screw device may comprise a Hamming code or other similar methods for error detection and correction that are known in the coding theory art. Additionally, data compression may be used in the coded indicia of the preferred embodiment. The present disclosure further includes unique device identification and information extraction through high data density structural encoding.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

While the specification concludes with claims particularly pointing out and distinctly claiming the present invention, it is believed that the present invention will be better understood from the following description in conjunction with the accompanying Figures, in which like reference numerals identify like elements, and wherein:

FIG. 1 illustrates a side perspective view of one embodiment of a readable portion positioned in an implant device in accordance with aspects of the present invention.

FIG. 2 illustrates a side perspective view of an alternative embodiment of a readable portion positioned in an implant device in accordance with aspects of the present invention.

FIG. 3 illustrates a side perspective view of an alternative embodiment of a readable portion positioned in an implant device in accordance with aspects of the present invention.

FIG. 4A illustrates a perspective view of one embodiment of the implant device of the present invention with a structurally encoded rod positioned therein.

FIG. 4B illustrates a perspective view of one embodiment of a structurally encoded rod.

FIG. 4C illustrates a perspective view of an alternative embodiment of a structurally encoded rod.

FIG. 4D illustrates a perspective view of an alternative embodiment of a structurally encoded rod.

FIG. 5A illustrates a perspective view of one embodiment of the implant device of the present invention with a structurally encoded rod positioned therein.

FIG. 5B illustrates a perspective and partially exploded view of one embodiment of the implant device of the present invention with a close up view of one embodiment of the structurally encoded rod.

FIG. 6A illustrates a perspective view of one embodiment of the implant device of the present invention with a structurally encoded rod positioned therein.

FIG. 6B illustrates a perspective and partially exploded view of an alternative embodiment of the implant device of the present invention with a close up view of one embodiment of the structurally encoded rod.

FIG. 7 illustrates an enlarged cross sectional view of one embodiment of a micromanufactured identifiable implant device in accordance with further aspects of the present invention.

FIG. 8A illustrates an example of a code database that can be used to code/decipher the surface modifications of the identifiable implant device of the present invention depicted in FIG. 7.

FIG. 8B illustrates a perspective view of the surface modifications of the database of FIG. 8A.

FIG. 9 illustrates a side perspective view of one embodiment of an identifiable implant device of the present invention.

### DETAILED DESCRIPTION

In the following detailed description of the preferred embodiment, reference is made to the accompanying drawings that form a part hereof, and in which is shown by way of illustration, and not by way of limitation, a specific preferred embodiment in which the invention may be practiced. It is to be understood that other embodiments may be utilized and that changes may be made without departing from the spirit and scope of the present invention.

The present disclosure relates to U.S. patent application Ser. No. 14/302,133, U.S. Pat. No. 9,101,321, U.S. patent application Ser. No. 14/302,197 and U.S. patent application Ser. No. 14/456,665, all of which are hereby incorporated by reference in their entirety.

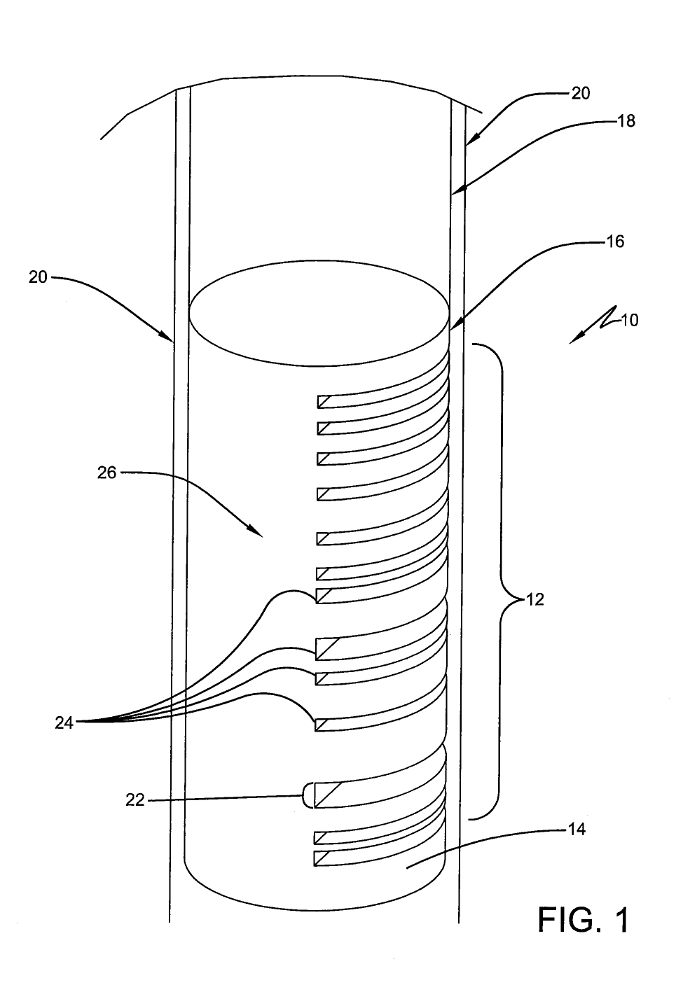

FIG. 1 illustrates a side perspective view of one embodiment of a readable portion positioned in an implant device in accordance with aspects of the present invention. More specifically, FIG. 1 shows an implantable rod structure 10 comprised of a readable portion 16 integrally formed with a main portion 18 and contained in an implant device 20. Alternatively, the readable portion 16 of the implant device 20 may be disposed upon the main portion 18. The readable portion 16 may be coupled to the main portion 18 by such means as fasteners, adhesive or through interference fit.

In this particular embodiment of the present invention, readable portion 16 has a series of notches 12 in one longitudinal side 14 of the readable portion 16. Each of the notches 12 is a modification to the surface of the readable portion 16, and has a predetermined width 22, though it may also have a predetermined, length, depth and/or shape each of which is capable of conveying additional encoded data. Further, each of notches 12 is located at a predetermined axial position 24 so as to create indicia 26 representing one-dimensional data, although it is contemplated that two and three-dimensional data can also be represented. In a preferred embodiment, rod structure 10 is a radiopaque structure, such as a tantalum rod. As will be further described below, rod structure 10 may also have a variable density such that rod structure 10 contains indicia in the form of a variable density internal structure or a particular mesh structure created by additive manufacturing, thereby increasing the density of the data coding. After implantation, rod structure 10 and indicia 26 are detectable and readable via any of a variety of imaging or measurement methods, such as x-ray, fluoroscopy, computed tomography, electromagnetic radiation, ultrasound, and magnetic resonance imaging. The indicia 26 may also be detected and received by conventional medical imaging devices. Imaging software, preferably high resolution imaging software, then reads and processes the data from the indicia 26 to decode and store and/or display the information from the implant device 20.

In a first embodiment of the present invention, the data represented by the indicia 26 on the surface of the rod structure 10 references unique information located in an external database. One example of such information includes data from the indicia 26 representing a unique numerical identifier corresponding to a wealth of manufacturer, patient, surgeon, or surgical procedure information located in an external healthcare facility database.

In further embodiments of the present information, the size of the indicia may be decreased, and the density of the data thereby increased, such that additional information beyond mere reference data may be recorded onto the surgical implant. Such embodiments are further discussed below. In the preferred embodiment of the present invention, error correction is used to increase the resolution of the imaging technology, thereby allowing an increase in data density. Error correction is discussed in more detail below.

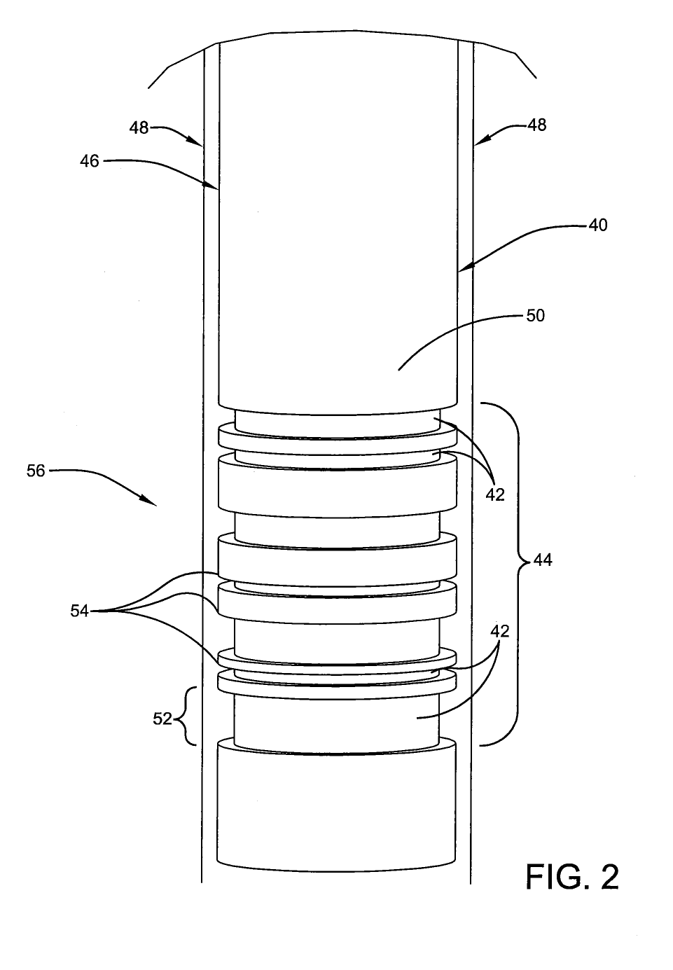

Referring now to FIG. 2, an implantable rod structure 40 of a preferred embodiment of the present invention features a series of notches 42 around the circumference of the rod structure 40. The implantable rod structure 40 of the preferred embodiment of FIG. 2 features a readable portion 44 shown in FIG. 2 to be integral with a main portion 46 and contained within an implant device 48. Alternatively, the readable portion 44 of the implant device 48 may be disposed upon the main portion 46. The readable portion 44 may be coupled to the main portion 46 by such means as fasteners or adhesives or through interference fit. Ideally, rod structure 40 will be comprised of a different material than implant device 48 so it is both visible and distinguishable in the imaging of the same. Each of the notches 42 is a modification to an exterior surface 50 of the readable portion 44, has a predetermined width 52 (and/or length, depth and/or shape), and is located at a predetermined axial position 54 so as to create indicia 56 representing one-dimensional data (or two or three-dimensional data, etc.). The rod structure 40 in the preferred embodiment is a radiopaque structure, such as a tantalum rod. After implantation, the rod structure 40 and indicia 56 are detectable and readable via a variety of imaging methods such as x-ray, fluoroscopy, computed tomography, electromagnetic radiation, ultrasound, and magnetic resonance imaging. The notches 42 of the preferred embodiment may be created using known lathe (machining) techniques or through additive manufacturing processes, as further discussed below. As opposed to indicia located only on a side of a rod structure as shown in FIG. 1, positioning of indicia 56 around the circumference of the rod structure 40, as shown in FIG. 2, increases visibility of the indicia 56 and readability of the data by various imaging methods. The indicia 56 is detected and received by medical imaging devices, which transmits the data to imaging software with sufficient resolution for accurately reading and interpreting indicia 56. The imaging software (not shown) reads the indicia 56 to decode and store and/or display the information from the implant device 48 on a user interface (not shown).

Although the indicia 26, 56 shown in FIGS. 1 and 2 respectively is oriented in a direction generally perpendicular to the axis of the rod structures 10, 40, the indicia 26, 56 of the rod structures 10 and 40 may also be oriented in a skewed or slanted orientation such that the indicia 26, 56 is not perpendicular to the axis of the rod structures 10, 40. As will be recognized by one having ordinary skill in the art, any embodiment of the exemplary rod structures shown in FIGS. 1-3 and 10 may include notches, screw threads, or similar surface modifications. Furthermore, each notch, thread, or similar surface modification may vary in depth, cross-section, or geometric shape across the series or array for further data storage possibilities.

In a preferred embodiment of the present invention, the data represented by indicia 56 on the surface 50 of rod structure 40 references unique information located in an external database. One example of such information includes the data from the indicia 56 representing a unique implant number corresponding to a wealth of manufacturer, patient, surgeon, or surgical procedure information located in an external healthcare facility database.

Error correction is used in a preferred embodiment of the present invention to increase the resolution of the imaging technology, thereby allowing an increase in data density for a given measurement technology. By encoding, for example, a number into the implant through micro-machined holes and/or notches, sufficient permutations of the code can be recorded. In a preferred embodiment of an implantable device according to the present invention, a tantalum marker used in polymer spine implants contains, for one example, 400 micron discrete notches. The full code width and the bit count could, in this example, be dictated by machining precision and accuracy, number of variable machining widths (e.g., 100 microns, 200 microns, and 300 microns), total bar length, and image resolution. To ensure robustness in the encoding scheme, error correction in the form of a Hamming code is implemented in the preferred embodiment but any error correction method known in the coding theory art could be employed. In the preferred embodiments shown in FIGS. 1 and 2, four variable width notches placed every 250 microns allow eight bits of data to be encoded reliably every millimeter and read by a computed tomography scan with sufficient resolution to identify the notches. This is an example under the preferred embodiment having values that are “power of 2 friendly” in order to clarify one embodiment of the present invention, though it will be appreciated by one of ordinary skill in the art that the power (i.e., number of possible combinations) can be increased with each additional variable. The specific values of any particular embodiment of the present invention depend upon the imaging and manufacturing resolution, which will improve over time, as one having ordinary skill in the art may recognize.

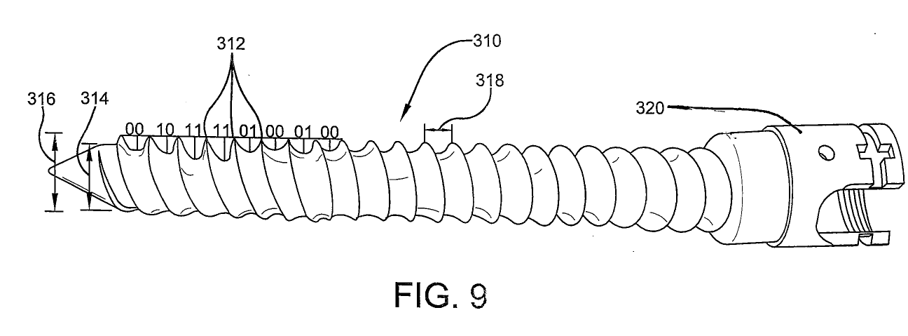

Referring now to FIG. 9, rod structure 310 includes a plurality of threads 312 in a spiral or helical configuration around the circumference of the rod structure 310. Although the threads 312 shown in FIG. 9 are continuous to form a screw structure, such as a pedicle screw, the inner diameter 314 on rod structure 310 between adjacent threads 312 is varied to form indicia. As indicated in FIG. 9, the predetermined indicia allow coded data to appear within the functional structure of the rod structure 310 before and after implantation. Alternatively, the outer diameter 316 of threads 312 may be varied in addition to, or instead of, the variation of the inner diameter 314 to retain coded indicia on the rod structure 310. Further, the axial spacing 318 between adjacent threads 312 may be varied in order to store data. Even further, the particular shape of the spacing between adjacent threads 312, such as a square, triangular, or circular shape, may also allow data storage in the rod structure 310. A variation of this embodiment includes a micromanufactured implant device having indicia in or on the head 320 of the rod structure 310, such as coded indicia in the head of a surgical screw.

Any of the embodiments of the present disclosure may include data relating to the unique image, properties, or manufacturing characteristics of the implant or component itself, such as particular programming language directed to identification or replication of the structure. Further, any of the embodiments, including each particular structure, disclosed in the present application may include encoded implant devices having the forms of, or being incorporated into, screws, rods, or other medical devices such as shoulder implants, hip implants, knee implants, or cardiovascular devices, stents, etc.

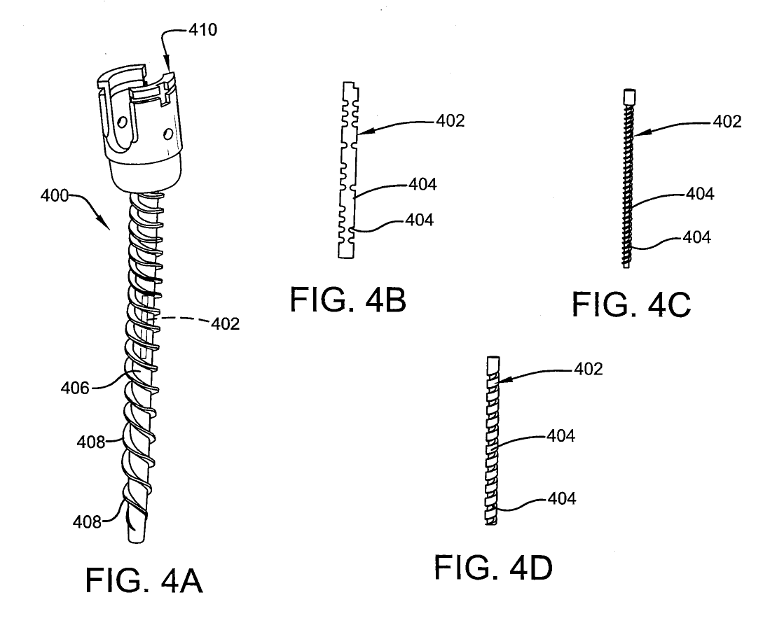

Reference is now made to FIGS. 4A-4D, which depict a cannulated titanium screw device 400 encoded with an implantable rod structure 402 (or implant body) having a series of notches 404 around the circumference of the rod structure 402 or a portion thereof. The cannulated screw device 400 of one embodiment of the present invention includes a cylindrical body component 406 (or screw member) that includes a plurality of threads 408 in a spiral or helical configuration around the circumference of the cylindrical body component 406. The plurality of threads 408 is continuous to form a screw structure. The cannulated screw device 400 further comprises a head 410 secured to an end of the cylindrical body component 406 or integrally formed therewith. The rod structure 402 is then inserted into the cannulated section of the cylindrical body component 406 before insertion of the screw device 400 into a pedicle or other part of the vertebrae, or any part of the body needing a screw device 400 or other bone fixation device. Each of the notches 404 is a modification to an exterior surface, has a predetermined width (and/or length, depth and/or shape), and is located at a predetermined axial position so as to create indicia representing one-dimensional data. The rod structure 402 in the preferred embodiment is a radiopaque structure, such as a tantalum rod. After implantation, the rod structure 402 and indicia are detectable and readable via a variety of imaging methods such as x-ray, fluoroscopy, computed tomography, electromagnetic radiation, ultrasound, and magnetic resonance imaging.

The notches 404 of the preferred embodiment may be created using known lathe (machining) techniques or through additive manufacturing processes, as further discussed below. As will be recognized by one having ordinary skill in the art, any embodiment of the exemplary rod structures 402 shown in FIGS. 4B-D may include notches, threads, or similar surface modification (shown as 404 in FIGS. 4B-D). Furthermore, each notch, thread, or similar structure may vary in depth, cross-section, or geometric shape across the series or array for further data storage. As opposed to indicia located only on a side of a rod structure as shown in FIG. 1, positioning of indicia around the circumference of the rod structure 402, as shown in FIG. 4, increases visibility of the indicia and readability of the data by various imaging methods. The indicia is detected and received by medical imaging devices, which transmits the data to imaging software with sufficient resolution for accurately resolving the indicia. The imaging software reads the indicia to decode and store and/or display the information from the screw device 400.

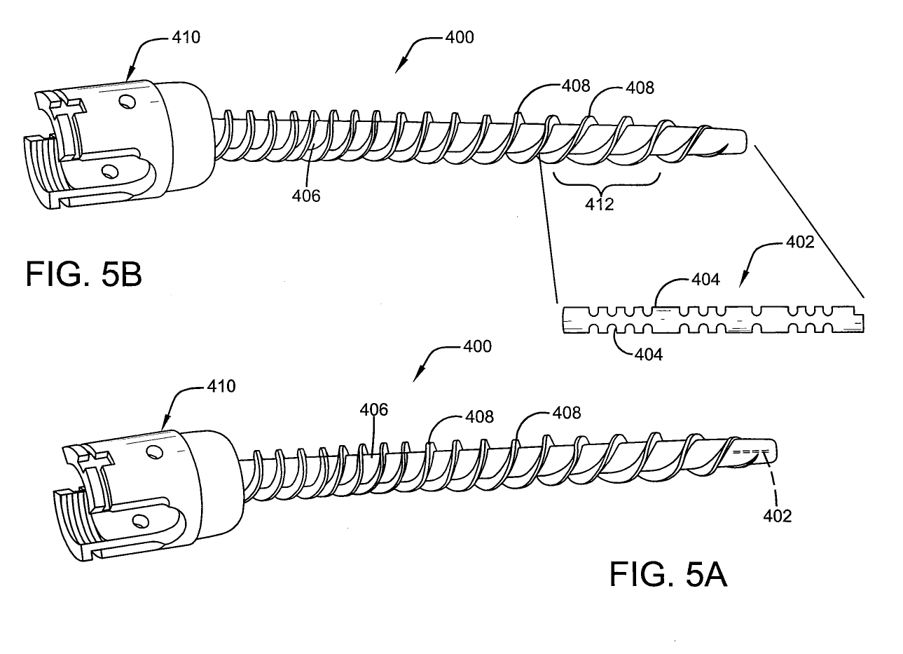

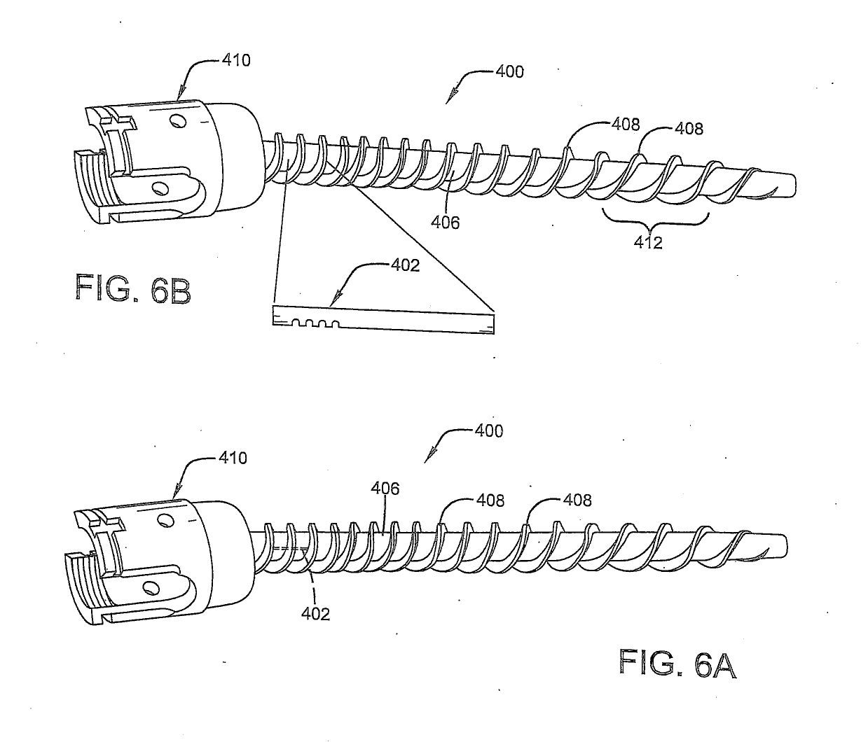

Referring now to FIGS. 5 and 6, the implantable rod structure 402 can be inserted into the cannulated section of the cylindrical body component 406 at the tip 412 of the screw device 400 or at the base 414 of the screw device 400 (or anywhere in between) depending on exterior factors, such as other hardware or implants and/or limitations of fluoroscopy. When the implantable rod structure 402 is inserted into the cannulated section of the cylindrical body component 406 at the base 414 of or at the tip 412 of the screw device 400, the indicia of the implantable rod structure 402 is viewable from any plane regardless of rotational orientation of the screw device 400.

Furthermore, in a preferred embodiment of the present invention. The implantable rod structure 402 is cannulated for use with minimally invasive surgeries (MIS) and other procedures as needed. When the implantable rod structures 402 are cannulated, the rod structures 402 should have a diameter bigger than 1 mm. Specifically, the MIS surgeries require a guide wire that needs to be threaded through the cannulated body of the rod structure 402 for placement of the screw device 400.

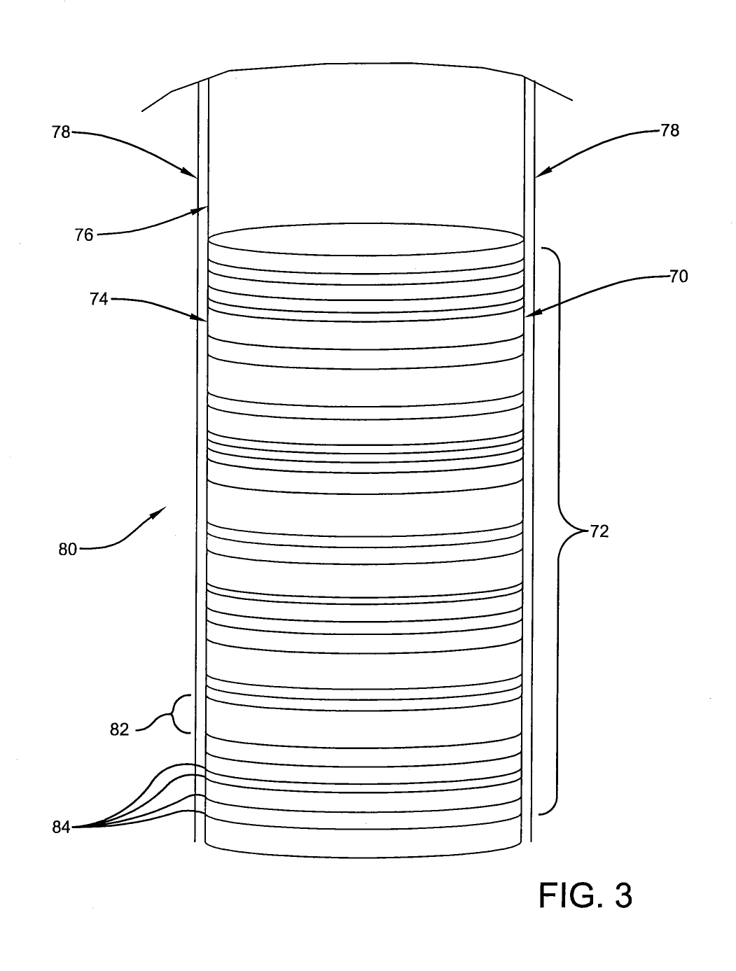

Referring now to FIG. 3, an implantable rod structure 70 of a preferred embodiment of the present invention features multiple materials in discrete layers 72 to create one-dimensional data around the circumference of the rod structure 70. The implantable rod structure 70 of the preferred embodiment of FIG. 3 features a readable portion 74 shown in FIG. 3 to be integral with a main portion 76 and contained within an implant device 78. Alternatively, the readable portion 74 may be disposed upon the main portion 76, or coupled to the main portion 76 by such means as fasteners or adhesives or through interference fit. Similar to the notched indicia shown in FIGS. 1 and 2, the variance of material across the layers 72 in the embodiment shown in FIG. 3 creates indicia 80 representing data that is readable across the axial dimension of the rod structure 70. Alternative embodiments may feature multiple material layers readable across a different dimension or a structure having a different shape constructed using layers of multiple materials.

The variation in material, as used in the embodiment of FIG. 3, includes a variation in composition. The composition of any material described in accordance with the present invention may include any physical or chemical characteristics of the material. As such, a variation in material includes a variation in any physical or chemical characteristics of the material.

Referring again to the preferred embodiment of FIG. 3, each of the distinct material layers 72 has a predetermined width 82 and is located at a predetermined axial position 84 so as to create the indicia 80 representing one-dimensional data. At least one of the layers 72 in the rod structure 70 of FIG. 3 is a radiopaque structure. In a preferred embodiment, each of layers 72 is composed of a particular material having some degree of opacity. Like the rod structures of FIGS. 1 and 2, after implantation, the rod structure 70 and indicia 80 of the implant device 78 of FIG. 3 are detectable and readable via a variety of imaging methods such as x-ray, fluoroscopy, computed tomography, electromagnetic radiation, ultrasound, and magnetic resonance imaging. The indicia layers 72 of the preferred embodiment shown in FIG. 3 are structured so as to be visible from any side of the rod structure 70 to increase readability of the data by imaging methods. The indicia 80 are detected and received by medical imaging devices, which transmits the data to imaging software, preferably high resolution imaging software. The imaging software reads the indicia 80 to decode and store and/or display the information from the implant device 78. The information or data encoded onto or into the implant devices of the embodiments disclosed in the present invention may be detected, decoded, read, transferred, stored, displayed, and/or processed.

The implantable device 78 of FIG. 3 may be manufactured using additive manufacturing (AM) techniques. Due to their precision and programmability, AM processes may be used for any of the embodiments shown in FIGS. 1-3 to allow a reduction in the size of the indicia and, therefore, increased density of data included onto the surface of the implantable rod structure. In some cases, machining may be sufficient to provide the indicia necessary for the implantable rod structure. With increased data density, additional information beyond mere reference data may be recorded onto the surgical implant 78. The data recorded onto the implant device itself may include the manufacturer, patient, surgeon, or surgical procedure information that would otherwise need to be stored in and accessed through an external database. Additionally, AM allows complex, mass customized, internal structures otherwise unavailable with conventional manufacturing, including three-dimensional structures discussed in further detail below. Moreover, AM eliminates the need for tooling and can therefore allow fabrication of implants with unique identifiers within the structure with no additional masks, molds, or user interaction.

ASTM International formed Committee F42 on Additive Manufacturing Technologies in 2009 with the mission of setting the standards for design, process, and materials with regards to AM. The committee defined a taxonomy of seven sub-technologies that together constitute the full suite of AM techniques.

Material extrusion is an additive manufacturing process where material is selectively dispensed through an extrusion nozzle. The most common implementation of this method involves the extrusion of thermoplastic material through a heated orifice. The material available for the most common implementation tends to be functional plastics that are sufficiently robust to withstand harsh environments such as chemical, mechanical, or temperature exposure.

Vat photo polymerization features a vat of liquid photo curable polymer that is selectively cured with an energy source such as a laser beam or other optical energy. The part is typically attached to a platform that descends one cure depth after a layer is completed and the process is repeated. This class of additive manufacturing benefits from feature sizes dictated by either the laser beam width or optical resolution in the X and Y axis and minimum cure depth in Z.

Powder bed fusion processes include selectively melting or sintering a layer of powder using an energy source such as a laser or electron beam, lowering the layer by a fabrication layer thickness, and adding a new powder layer by delivery with a rake or roller and material storage mechanism. The process continues with the next layer. Un-melted powder in the bed acts inherently as support material for subsequently built layers.

Material jetting uses ink-jetting technology to selectively deposit the build material with a cure prior to the application of subsequent layers. An exemplary version of this technology may be ink-jetting multiple photo-curable polymers and following the inkjet head with a UV lamp for immediate and full volume curing. With multiple materials, fabricated items can be multi-colored or materials can be chosen with varying stiffness properties. Ink-jetting is also naturally well suited for parallelism and thus can be easily scaled to larger and faster production.

Binder jetting includes selectively ink-jetting a binder into a layer of powder feedstock. Additional powder material is then dispensed from a material storage location by a rake or roller mechanism to create the next layer. Some binder jetting technologies may require a post-anneal furnace cycle depending on the materials being used (e.g., metals, ceramics). One exemplary system may inkjet color (much like a commercial inkjet color printer) in addition to the binder into a powder, and may therefore provide structures with colors throughout the structure for conceptual models. Another binder jetting system may utilize a post anneal process to drive out the binder to produce metal or ceramic structures.

Sheet lamination is another additive manufacturing process in which individual sheets of material are bonded together to form three-dimensional objects. In one exemplary embodiment, sheets of metal are bonded together using ultrasonic energy. The process has been shown to produce metallurgical bonds for aluminum, copper stainless steel, and titanium. A subsequent subtractive process between layers adds internal structures and other complex geometries impossible with conventional subtractive manufacturing processes that start from a billet of material.

Directed energy deposition is another additive manufacturing process that directs both the material deposition and the energy source (typically a laser or electron beam) at the surface being built. Directed energy deposition processes typically use powder or wire-fed metals and exemplary applications of the process may include repair of high value components used in aircraft engines.

The implant device of the present invention may also be manufactured by conventional methods such as a machining operation using any milling, lathe, or drilling operation to include standard machining and fabrication methods known in the art of manufacturing medical implants.

The embodiments of FIGS. 1-3 show an implantable rod structure having a length of one centimeter. Exemplary embodiments of each implant device shown in FIGS. 1-3 include each notch or material variation having a thickness of between 0.1-0.3 millimeters inclusive, which results in storage of about 30-40 bits of information on the implantable rod structure. After utilizing bits for Hamming code error correction, about 25-35 actual data bits create approximately 30 million to 30 billion indexing options into an external database or for limited information stored on the implant such as an implant expiration date and lot number.

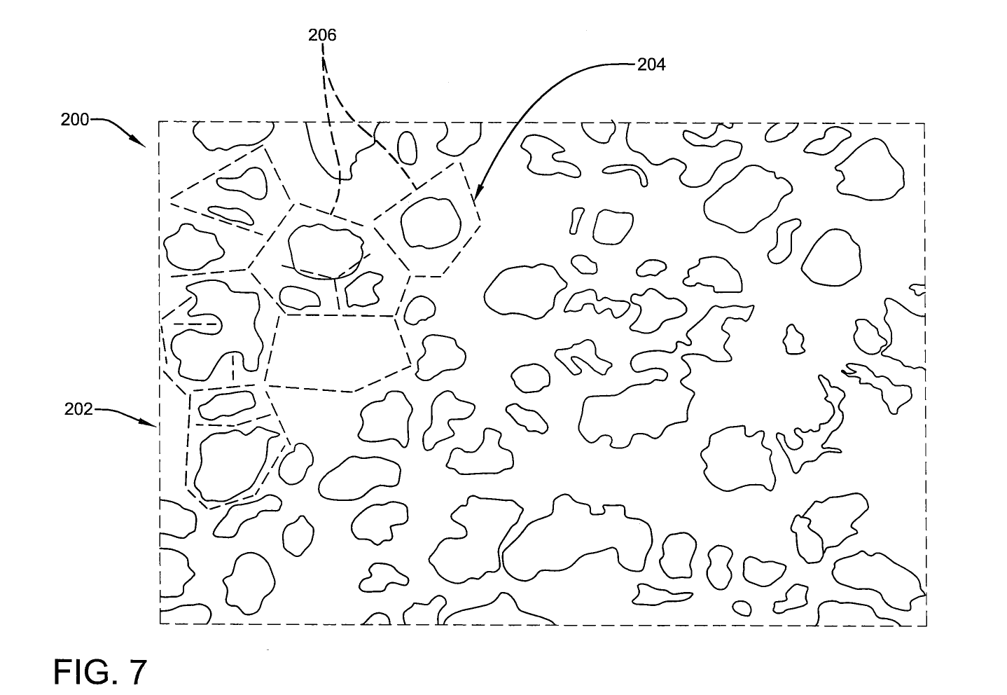

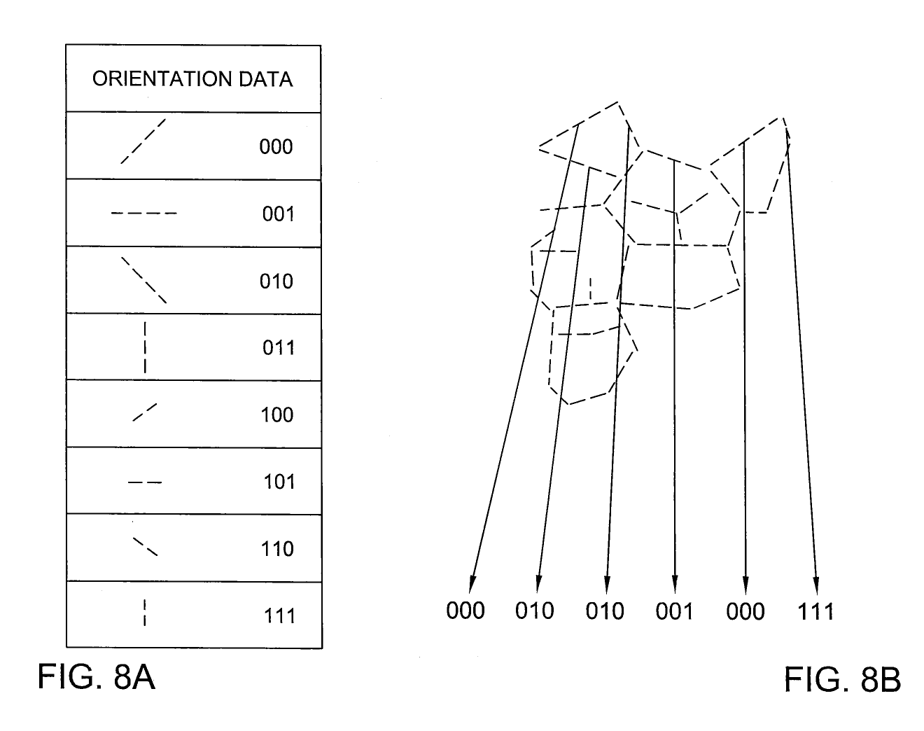

Reference is now made to FIG. 7, which shows an implantable device 200 of a preferred embodiment of the present invention. The implantable device 200 of the preferred embodiment is a metal mesh structure fabricated using additive manufacturing (also known in the art as 3D printing). Through an AM manufacturing process, a unique internal structure is formed while maintaining the structural requirements of the implant device 200. A readable portion 202 includes an internal structure 204 inside the readable portion 202. The internal structure 204 includes linking structures 206 (in dashed lines) that interconnect to form the internal structure 204. Individual linking structures 206 in the preferred embodiment shown in FIG. 7 each have a predetermined size and orientation in reference to a unique registration structure that would be included in every implant and easily identifiable. As shown in FIGS. 8A and B, the size and orientation of a particular linking structure 206 of the preferred embodiment of the present invention is predetermined to represent binary data and is linked to a database, such as the database illustrated in FIG. 8A. As with the embodiments of the present invention shown in FIGS. 1-6, the encoded data is read to gather valuable information relating to the implant, patient, surgical operation, etc. The data contained in the readable portion 202 of the implantable structure 200 can be accurately read through non-invasive means such as x-ray, fluoroscopy, computed tomography, electromagnetic radiation, ultrasound, and magnetic resonance imaging. FIGS. 7-8 show, in detail, the readable portion 202, internal structure 204, and linking structure 206 of the implantable structure 200 according to one embodiment of the present invention.

One or more of the embodiments of the present invention are structurally encoded devices, which refers to the 3D encoding of digital information in a structure as variations in geometric or physical features—widths, densities, color, feature angles, etc. Bar codes are an example of a 2D encoding of digital information with modulations of color (dark versus light) with varying widths of printed bars on a surface. A typical embodiment of the structurally encoded devices of the present invention may contain data that is not readily apparent to a viewer of the device structure to preserve patient privacy. Further, encoding of the typical embodiments of the present invention is handled by physical means other than those accomplished through circuitry, electromagnetic or other, within the implant device itself or through a type of internal storage means such as magnetic storage means or the like. Such structurally encoded devices, as disclosed herein and described in relation to the typical and/or preferred embodiments of the present invention allow simplified production, maintenance, and/or operation costs for identification, storage, and/or retrieval of unique implant data while retaining a substantial amount of information with reduced probability for error.

The preferred embodiments of the present invention, as shown individually in FIGS. 1-9, may be manufactured by one or more of the AM processes described above. The method of manufacturing and identifiable implant according to a preferred embodiment of the present invention comprises providing a main portion of an identifiable implant device, providing a readable portion of an identifiable implant device, printing a first material onto a first readable portion surface to create a first printed layer, and printing a second material onto the first printed layer to create a second printed layer, wherein the first material and the second material can be the same or different. At least one of the printing of the first material onto the first readable portion surface and the printing of the first material onto the first printed layer comprises printing encoded indicia. Further, the encoded indicia may comprise volumes of a second material having different density than the first material found elsewhere in the readable portion of the identifiable implant device. As an example, the readable portion of an identifiable implant may be formed by an AM or 3D printing process such that micro-volumes of a metal material having a relatively high density are deposited within a polymer substrate having a relatively low density. Other combinations that would include any combination of metal, polymer, ceramic, or composites, such as carbon fiber or carbon nanotubes, may be used. Additionally, any single or combination of composite or nanoparticle material, including fine particles between 1 and 100 nanometers in size, may be used for the present structure, such as the readable portion. Then encoded indicia may also comprise voids in the first material of the identifiable implant device. Further, any single embodiment of the present invention may be manufactured using a combination of traditional manufacturing processes and additive manufacturing processes. For example, a 3D printed implant device with internal indicia formed by the 3D printing process may also have a series of notches micromachined onto an exterior surface of the 3D printed implant device.

The identifiable implant device of the present invention enables more accurate reporting, reviewing, and analyzing of adverse event reports so that problem devices can be identified and corrected more quickly. Additionally, the identifiable implant device of the present invention reduces medical error by enabling health care professionals and others to rapidly and precisely identify a device and obtain important information concerning the characteristics of the device. The present invention enhances analysis of devices on the market by providing a standard and clear way to document device use in electronic health records, clinical information systems, claim data sources, and registries. Through the identifiable implant device of the present invention, a more robust post-market surveillance system may also be leveraged to support premarket approval or clearance of new devices and new uses of currently marketed devices. The present invention further provides a standardized identifier that will allow manufacturers, distributors, and healthcare facilities to more effectively manage medical device recalls. Moreover, the present invention provides a foundation for a global, secure distribution chain, helping to address counterfeiting and diversion and prepare for medical emergencies. The identifiable implant device of the present invention enables development of a medical device identification system that is recognized around the world.

Other variations are within the spirit of the present invention. Thus, while the invention is susceptible to various modifications and alternative constructions, a certain illustrated embodiment thereof is shown in the drawings and has been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An implant device identifiable after implantation comprising:
a cannulated body component; and
an implant body disposed within the cannulated body component and defining a plurality of planes, wherein said plurality of planes comprises separately readable indicia, and further wherein the separately readable indicia can be read from at least two different directions with respect to said implant body.

2. The implant device of claim 1, wherein said separately readable indicia are discernible by at least one of x-ray, fluoroscopy, computed tomography, electromagnetic radiation, ultrasound, and magnetic resonance imaging.

3. The implant device of claim 1, wherein said separately readable indicia comprises information in the form of a code.

4. The implant device of claim 3, wherein said code comprises error correction coding.

5. The implant device of claim 3, wherein said code comprises a Hamming code.

6. The implant device of claim 1, wherein said implant body comprises a rod.

7. The implant device of claim 6, wherein said rod comprises a tantalum rod.

8. A screw device identifiable after implantation comprising:
a screw member comprising a cannulated body component and a head secured to an end of the cannulated body component; and
an implant body disposed within the cannulated body component of the screw member and defining a plurality of planes, wherein each of said planes comprises separately readable indicia.

9. The screw device of claim 8, wherein said separately readable indicia are discernible by at least one of x-ray, fluoroscopy, computed tomography, electromagnetic radiation, ultrasound, and magnetic resonance imaging.

10. The screw device of claim 8, wherein said implant body comprises a rod.

11. The screw device of claim 10, wherein said rod comprises a tantalum rod.

12. The screw device of claim 8, wherein said separately readable indicia include an array of voids on or in a corresponding plane of said implant body.

13. The screw device of claim 8, wherein said separately readable indicia include an array of embedded markers on or in a corresponding plane of said implant body, and further wherein said array of embedded markers comprises a modulation of material composition such that a first material composition of a first one of the array of embedded markers is different than a second material composition of a second one of the array of embedded markers.

14. The screw device of claim 8, wherein said separately readable indicia include an array of embedded markers on or in a corresponding plane of said implant body, and further wherein said array of embedded markers comprises a modulation of material density such that a first material density of a first one of the array of embedded markers is different than a second material density of a second one of the array of embedded markers.

15. The screw device of claim 8, wherein said separately readable indicia include a first embedded marker on or in a first plane of said implant body and a second embedded marker on or in a second plane of said implant body, and further wherein the material composition of the first embedded marker if different from the material composition of the second embedded marker.

16. A screw device identifiable after implantation comprising:
a screw member comprising a cannulated, cylindrical body component and a head secured to an end of the body component configured for implantation into a pedicle of a vertebrae; and
an implant body disposed within the cannulated, cylindrical body component of the screw member and comprising at least one rod and indicia around an entire circumference of said at least one rod, and wherein said indicia are discernible by at least one of x-ray, fluoroscopy, computed tomography, electromagnetic radiation, ultrasound, and magnetic resonance imaging.

17. The screw device of claim 16, wherein said indicia comprise an array of notches.

18. The screw device of claim 17, wherein said array of notches forms at least one bar code.

19. The screw device of claim 18, wherein said at least one bar code comprises error correction coding.

20. The screw device of claim 16, wherein said at least one rod is a tantalum rod.
